(19) United States
(12) Patent Application Publication
(10) Pub. No.: US 2009/0036528 A1
(43) Pub. Date: Feb. 5, 2009

(54) Multifunctional and combinational application of aspartame and or futhan

(76) Inventors: Tony E. Hugli, San Diego, CA (US); John E. Adams, Nichols Hills, OK (US)

Correspondence Address: Martin G. Ozinga;Phillips Murrah, P.C., Corporate Tower, 13th Floor, 101 N. Robinson Ave., Oklahoma City, OK, 73102, US

(21) Appl. No.: 12/220,949

(22) Filed: Jul. 30, 2008

Related U.S. Application Data
(60) Provisional application No. 60/964,987, filed on Aug. 16, 2007.
(60) Provisional application No. 60/962,651, filed on Jul. 31, 2007.

(51) Int. Cl. A61K 31/24; A61P 17/00; A61P 19/10

(52) U.S. Cl. 514/542

(57) ABSTRACT

The present invention is an application, composition, and method of using a pharmaceutically effective amount of aspartame or its primary metabolite aspartyl-phenylalanine in systematic and periodic application or dose as an aspirin (NSAID) substitute, treatment for osteoporosis, and or topical treatment for Rosacea.

### CROSS-REFERENCE TO RELATED APPLICATIONS

Priority is claimed from provisional patent applications U.S. Ser. No. 60/964,987, filed on Aug. 16, 2007, and U.S. Ser. No. 60/962,651, filed on Jul. 31, 2007 and incorporated by reference herein.

### BACKGROUND OF THE INVENTION

1. Field of the Invention

In general, the present invention is an application, composition, and method of using the dipeptide ester aspartame. More in particular, the present invention is a systematic and periodic application of aspartyl-phenylalanyl methyl ester, hereinafter referred to generally as aspartame, as a topical formulation and/or an oral formulation for applications utilizing aspartame beneficial analgesia, anti-inflammatory, osteoporosis, anti-platelet blood thinning effects and combinations thereof. It is understood that the current invention may provide a pharmaceutically effective amount of aspartame or its primary metabolite aspartyl-phenylalanine as an aspirin (NSAID) substitute as well as enhance calcium solubility, bio-availability, absorption, mineral transfer and bone growth for animals and humans as will be described in greater detail below. It is also contemplated a pharmaceutically effective amount of aspartame may be utilized for a topical application for rosacea in specific.

2. Description of the Prior Art

The dipeptide ester aspartame (N-L-alpha-aspartyl-L-phenylalanine 1-methyl ester) is a well known food additive used primarily as a food supplement and sweetener. It is sold under various trade names including the name EQUAL. It has been recognized that aspartame has additional multiple biological and physiological properties including an analgesia or pain relief. See U.S. Pat. Nos. 5,998,473 and 5,654,334. Aspartame also is known to reduce blood viscosity resulting in blood thinning as documented by increased bleeding times in man and animals that ingested proper quantities of this compound. See U.S. Pat. No. 6,919,374.

There have been numerous advancements in the understanding and use of aspartame. The above discussed prior art is not intended to be remotely exhaustive, but generally as background. The current invention provides an inexpensive and effective application, composition and method of using aspartame not currently found in the known art.

### SUMMARY OF THE INVENTION

In view of the foregoing disadvantages inherent in the known types of compositions and methods of using aspartame in the prior art, the present invention provides a new and improved effective application, composition, and method of using the same where the prior art fails. As such, the general purpose of the present invention, which will be described subsequently in greater detail, is to provide a new and improved application, composition and method of using aspartame and or FUTHAN which has all the advantages of the prior art and has reduced or none of the disadvantages.

To attain this, the present invention essentially comprises a systematic and periodic application of a topical or oral formulation that includes a pharmaceutically effective amount of aspartame for use such as, but not limited, to general analgesic applications, topical applications, bone growth, density, and maintenance applications, as well as chronic or sickle cell anemia applications.

It is understood that the current invention may provide a pharmaceutical effective amount of aspartame or its primary metabolite aspartyl-phenylalanine as an aspirin (NSAID) substitute as well as enhances calcium solubility, bioavailability, absorption, mineral transfer and bone growth.

Furthermore, it is contemplated that the current invention may comprise a systematic and periodic application of a topical or oral formulation that includes a pharmaceutically effective amount of aspartame and/or FUTHAN for the treatment of Rosacea.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in this application to the details of construction, arrangement of the components, and amounts thereof set forth in the following description. The invention is capable of other embodiments and of being practiced and carried out in various ways. Also, it is to be understood that the phraseology and terminology employed herein are for the purpose of description and should not be regarded as limiting. As such, those skilled in the art will appreciate that the conception upon which this disclosure is based may readily be utilized as a basis for the designing of other compositions, methods, and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Therefore, it is an object of the present invention to provide a new and improved application, composition and method of using aspartame that may be easily and effectively used for bone growth, density, and maintenance.

It is a further object of the present invention to provide a new and improved application, composition and method of using aspartame for topical applications as an analgesic and anti-inflammatory for numerous dermal utilities.

An even further object of the present invention is to provide a new and improved application, composition and method of using aspartame for osteoarthritis.

Still another object of the present invention is to provide a new and improved application, composition and method of using aspartame and or FUTHAN which provides all of the advantages of the prior art, while simultaneously overcoming some of the disadvantages normally associated therewith.

Another object of the present invention is to provide a new and improved application, composition and method of using aspartame for the safe and reliable treatment of various oral pain and soreness.

Yet another object of the present invention is to provide a new and improved application, composition and method of using aspartame that is commercially available such that public awareness is garnered and those desiring the beneficial affects will have a viable and readily available application, composition, and method of using the same.

An even further object of the present invention is to provide a new and improved application, composition and method of using aspartame that combines proven and or active ingredients that have already passed F.D.A. approval as well as known delivery compounds.

Still another object of the present invention is to provide a new and improved application, composition and method of using aspartame that is susceptible to a low cost of manufacture with regard to ingredients and associated labor of producing same, and, thus accordingly, is then susceptible to low prices of sale to the consuming public thereby making such economically available.

Yet another object of the present invention is to provide a new and improved application, composition and method of using aspartame as an aspirin substitute with all the beneficial qualities and with no or reduced side effects associated with aspirin.

It is also an object of the present invention to provide a new and improved application, composition and method of using aspartame that may generally thin blood and thereby reduce stroke and heart attacks.

These, together with other objects of the invention, along with the various features of novelty, which characterize the invention, are pointed out with particularity in the claims annexed to and forming a part of this disclosure. For a better understanding of the invention, its operating advantages, and the specific objects attained by its uses, reference should be had to the accompanying descriptive matter in which there are illustrated preferred embodiments of the invention.

### BRIEF DESCRIPTION OF THE PICTORIAL ILLUSTRATIONS, GRAPHS, DRAWINGS, AND APPENDICES

The invention will be better understood and objects other than those set forth above will become apparent when consideration is given to the following detailed description thereof. Such description makes reference to the annexed pictorial illustrations, graphs, drawings, exhibits and appendices herein.

FIG. 1 is a depiction in accordance with a preferred embodiment of the invention.

FIG. 2 is a depiction in accordance with a preferred embodiment of the invention.

FIG. 3 is a depiction in accordance with a preferred embodiment of the invention.

FIG. 4 is a depiction in accordance with a preferred embodiment of the invention.

### DETAILED DESCRIPTION

The current invention is a utilization of a pharmaceutically effective amount of aspartame. It is understood that the following description of application, components, and methods of using the same is not limited to exact percentages, quantities, or ingredients and that it is understood equivalent ingredients known in the art may be substituted or added. It is further contemplated to utilize FUTHAN.

### Overview

The current invention has been utilized, by means of example, with that pigs fed 75-300 mg aspartame/100 weight/day over a two month period, which significantly increased bleeding times and elevated platelet counts, leading to these cells having reduced adhesion properties in the vasculature. Recent chemical evidence has documented the mechanism of the analgesia and blood thinning properties of ASP as attributable in part to the compound's ability to inhibit the cyclo-oxygenase enzymes known as COX-1 and COX-2. It is contemplated that only the intact aspartame molecule and not the dipeptide Asp-Phe exhibits analgesic properties, then methods of delivery may need to take into account ester hydrolysis (i.e. in vivo conversion of Aspartame to Asp-Phe). It is contemplated that a preferred embodiment may use a coated tablet or pill that may prevent or reduce hydrolysis of the aspartame ester group in the acidic conditions of the stomach.

The COX enzymes are responsible for the production of a number of bioactive products called prostaglandins from a common cellular and membrane lipid called arachidonic acid. Certain of the prostaglandins (PGs) contribute to the pain response and others influence platelet aggregation and blood clotting phenomena. These are the same mechanisms, functions, and properties that are commonly associated with the natural analgesic aspirin. In this regard, aspartame is a functional aspirin substitute (i.e. new/novel NSAID), of nearly the same potency and efficacy. However, aspartame can not cause the covalent chemical modification of acetylation that aspirin does, thus avoiding potentially harmful side effects commonly associated with chronic aspirin use. Aspartame also avoids the allergic sensitivity response to aspirin, a relatively common side effect.

The chemical properties of aspartame suggest that it may be capable of binding or carrying divalent metal ions or cations (i.e. minerals such as calcium, magnesium, zinc, iron, copper and other essential trace divalent metal ions). This hypothesis was tested experimentally using the technique of mass spectroscopy (MS) to determine if the apparent mass or molecular weight of either ASP at 294 mass units (mu) or the free dipeptide Asp-Phe at 280 mu was altered in the presence of calcium ions (molecule weight of 40 mass units). Separation of aspartame and Asp-Phe on a carbon-18 column using reverse phase solvent elution demonstrated that these compounds were authentic and relatively pure. The MS results/data indicate no change in the mass of aspartame in the presence of calcium chloride salt at a molar ratio of 5:1 (Calcium:ASP). However, both monomers and dimers of the free peptide Asp-Phe DID bind one (1) calcium ion evidenced by signals of a calcium:Asp-Phe complex at 319 mass units (Asp-Phe monomer at 280 mu, minus one proton, plus 40 mu for a calcium ion) and a calcium: 2×Asp-Phe complex at 559 mu (Asp-Phe dimer at 560 mu, minus one proton, plus 40 mu for a calcium ion). Therefore, evidence shows that Asp-Phe binds calcium ions and by implication has an ability to bind other divalent minerals as well.

The chemical evidence demonstrating calcium binding to Asp-Phe, the dipeptide metabolite of aspartame, indicates the potential role of the aspartame-derived dipeptide in enhancing calcium (mineral) availability, calcium (mineral) solubility and calcium (mineral) absorption. It is reported that calcium solubility, bio-availability and absorption are not necessarily linked. Certain calcium salts are much more soluble than others (e.g. calcium bis-glycinate is 200 time more soluble than calcium citrate, and that calcium citrate is many times more soluble than calcium carbonate or calcium phosphate, and yet the bio-availability of these salts ranges between 23% and 37% (J. of Amer. College of Nutrition (vol. 19) No. 90002, 119S-136S, (2000) American College of Nutrition.). Absorption is the key variable in calcium bioavailability, and calcium absorption is markedly enhanced when taken during meals or food intake because the increased levels of stomach acid increases solubility of certain calcium salts.

It is contemplated that calcium binding to the dipeptide Asp-Phe enhances calcium solubility in the intestine, and enhances/increases calcium transfer to blood, tissues and cells. Asp-Phe may enhance absorption of other nutritionally important metal cations such as magnesium, iron, zinc, copper, and essential trace metal ions. Therefore, Asp-Phe may enhance metal ion transport and absorption. It is well known that excessive calcium absorption can lead to depletion or reduction in absorption of magnesium and other important minerals. See Calcium Supplements: Practical considerations. Heaney R. P. Osteoporosis International. February 1991. It is also contemplated that Asp-Phe reduces the need for ingesting excessive levels of calcium to enhance absorption and prevent imbalances in mineral absorption. Consequently, it is further contemplated ingesting Aspartame may make the entire process of ‘passive’ calcium transfer, i.e. absorption, from the digestive system to the circulation more efficient, and thereby promote mineral distribution to the cells and tissues including bone.

In addition, aspartame (or the metabolite Asp-Phe) can facilitate the ‘active’ transfer of bound minerals such as calcium, magnesium and other nutritionally essential metal ions from the digestive system into tissues and cells. Cells/tissues maintain a facilitative cellular transport system for key nutrients such as amino acids and dipeptides (Clin. Pharmacol. Ther. 63: 580-93, (1998)). It is known that protein degradation often stops at the dipeptide level because there are few enzymes that can degrade dipeptides efficiently. It is also known that a membrane transporter system exists for small peptides as well as free amino acids. A dipeptide like Asp-Phe can be actively transported by a receptor-specific transporter process through the intestinal wall to the lymphatic and blood vascular systems for distribution throughout the body. Similar transporter processes exist for cellular elements that compose most tissues including bone. This is an additional ‘active’ mechanism, i.e. facilitative transport, for delivering nutritional minerals bound to peptides and amino acids to cells and tissues. It is known that insulin enhances amino acid/peptide transport and thus diabetics may benefit from Aspartame/Asp-Phe ingestion, but only via passive transport mechanisms without added insulin. Therefore, it is contemplated that ingestion of a pharmaceutically effective amount of aspartame/Asp-Phe will result in ‘active mineral transport’ leading to elevated bioavailability, absorption and transport of minerals, higher blood levels of soluble or useable minerals, and enhanced utility of minerals in muscles, tissues and bone.

It has been reported that isotopically labeled Aspartame/Asp-Phe distributes to various tissues including the bones (See Aspartame Physiology and Biochemistry, Ed. Stegink, Lewis, D. and Filner, L. J. (1984), Marcel Decker, NY, Chapter 8, “Tissue Distribution of Orally Administered Isotopically Labeled Aspartame in Rat.” By Matsuzawa, Y., O'Hara, Y., pp. 161-199.). It is well established that the ester group on aspartame is labile to both acid hydrolysis, as exists in the stomach, and to enzyme esterases that are common to all body fluids and tissues. Therefore, a significant portion of the aspartame will be converted to the free dipeptide Asp-Phe after ingestion and the free dipeptide portion of the ingested aspartame (Asp-Phe) has been shown to bind divalent metal ions, i.e. minerals such as calcium. It is contemplated that this mechanism will enhance the transfer of essential minerals from the digestive system to the blood, tissues, and cells. Further evidence that aspartame (Asp-Phe) ingestion contributes to muscle (tissue) density and bone density enhancement comes from mouse studies. The SRT-ORT mouse model is a genetically deficient mouse strain obtained from NIH with a natural history of developing osteoarthritis. Blinded experiments were conducted with littermates divided into two groups, one fed chow containing 4 mg Aspartame/g. and the other fed non-supplemented chow over a period of 12-18 months. X-ray analyses were performed on the cortex and marrow spaces of the right and left femurs, as well as the quadriceps in the femoris area and joint calcification. The results show an increase in cortical bone density for the treated animals with a P value of 0.0058. The increase in calcification in the treated animal cohort at the injured joints gave a P value <0.0001. An unexpected increase in quadriceps muscle mass index for the treated animals gave a P value of 0.0185. These analyses were preformed on an n value of 23 animals in the treated set and 18 animals in the controls. Therefore, it is contemplated that animals fed aspartame over an extended time period have increased bone density and muscle mass indicating increased or enhanced calcium, and perhaps other minerals, bioavailability and utility.

Supportive anecdotal data now exists from a limited number of human subjects. There is evidence for bone density increase from dental records in a male subject who ingested levels of Aspartame in the range of 150-300 mg/day over several years. Another 64-year-old female subject that has ingested in excess of 300 mg/day for several years has bone scans that show a lumbar spine bone density (L1-L4) of 1.74 g/cm2, which is 148% the density of a young adult The density of the right and left femur were also elevated with a mean of 1.2 g/cm2, which is 122% the density of a young adult. See FIG. 34. This subject is particularly unusual in that she is a breast cancer survivor taking various drugs that are known to interfere with calcium utility in addition to being at an age that would predict bone loss. It is contemplated that ingestion of aspartame (or Asp-Phe) at proper levels can increase bone density, growth and development in man and animals. Normally, enhanced calcium adsorption and bioavailability leads to bone maintenance and growth but not enhanced bone density in grown adults unless the bone cells are somehow stimulated. Therefore, we conclude and claim that either aspartame or the metabolite Asp-Phe is a bone cell stimulant resulting in bone density enhancement.

Consequently, the increase in the blood level of Asp-Phe from hydrolyzed or de-esterified ASP leads to an increase in blood levels of bio-available, useable and soluble calcium and other minerals. The common mineral nutrients calcium phosphate and calcium carbonate are relatively insoluble salts limiting the level of free or physiologically useable calcium in the blood to approximately 3-5 millimolar (mM). During conditions of nutritional or physiological stress, the usable or free calcium levels may drop by 50% or more. Calcium levels below 2.5 millimolar can cause Tetany. When the useable blood calcium levels are low, the physiological condition is called ‘Calcium Stress’. This condition is often present during late stage pregnancy, birthing and lactation in man and animals. It is also a common condition during a number of chronic and acute disease processes including certain cancers and diabetes. Calcium or mineral ion stress may become particularly acute during clinical treatment for diseases, and may be brought on by drugs that deplete certain mineral levels or reduce normal utilization of minerals like calcium. Another common physiologic condition where short-term mineral ion stress can be significant is during exercise, physical exertion and athletics.

These findings and documentations that Asp-Phe can bind or carry calcium (mineral) ions to or in the blood indicates that this compound or the precursor aspartame may be used to treat or correct the condition of “calcium stress,” which is currently unknown in the prior art. The levels of aspartame that may or must be ingested to produce enhanced calcium bio-availability/absorption are higher than those used to sweeten foods or drinks. Assuming that the calcium levels fall to 50% of normal during stress (i.e. 2.5 mM blood calcium), then as much as 0.3-0.6 mM ASP might be required to provide significant or detectable relief (i.e. 10-20% increase in useable calcium). This is in the range of 274 mg/mM×0.3-0.6 mM=82-164 mg/liter of blood. Since the average adult man has 8 liters of blood, the daily-required dose of ASP to prevent or treat calcium stress could be in the range of 650 mg to 1.3 g. of ASP per day.

It is contemplated that aspartame represents a dual functioning therapeutic, having two proven, specific and independent functions and activities. The proven physiologic effects and mechanisms of aspartame relate directly to two major pathologic and health conditions in man and or animals. The aspirin-like analgesic activity of aspartame subsequently affects or reduces the incidence of stroke and heart attacks because of the reduced blood viscosity or thinning effect that takes place as a result of COX enzyme inhibition. The proven effects of aspartame or its metabolite Asp-Phe on bone development, maintenance and growth represents an inherent dual function of this compound beneficial to aiding normal bone development and in prevention and treatment of bone diseases.

It is therefore contemplated that the current invention may utilize aspartame with numerous active ingredients for numerous products, applications, and utilities as described herein. Some of these may be Acyclovir (an active ingredient in antiviral creams for treatments of shingles, cold sores, etc.) Calamine, Zinc Oxide, Benzocane, Resorcinol, Lidocaine Hydrochloride, Benzalkonium Chloride, Campho, Phenol, Terbinafine hydrochloride, Miconazole Nitrate, Padamate, Clotrimazole, Menthol, Thymol, Methyl salcylate, various mineral compounds, such as calcium and various vitamins such as A, D, E, and so forth.

### Bone Maintenance/ Calcium Availability

There are many treatments and products that claim to maintain bone, but few agents can enhance bone growth. The current invention utilizing aspartame can both maintain bone and enhance its density, strength and growth. It is contemplated, therefore, that a preferred embodiment of the invention may utilize an effective pharmaceutical amount of aspartame to enhance bone growth and maintenance. Furthermore, the utility of the current invention has been supported by human X-ray and bone scan data, as well as animal bone studies in mice and pigs.

The mechanism of the action is contemplated due to the ability of the dipeptide/dipeptide ester to bind metal ions essential in the growth and maintenance of bone structure, namely calcium, zinc and magnesium. The process of increasing blood metal ion levels, i.e. enhancing soluble or useable calcium levels, by binding to aspartame is a physiologic benefit with important health consequences. Because common nutritional calcium salts, mainly calcium phosphate and calcium carbonate, are relatively insoluble in blood and body fluids, this may limit the ability of calcium to be incorporated into bone.

A second action of the aspartame compound, or the free dipeptide metabolite, may occur on the bone cells themselves, i.e. the osteoblasts or osteoclasts. This may occur either via direct action or by indirect action such as through factors called prostaglandins known to exert an effect on bone tissue. It is also contemplated the nature of the molecular structure of the dipeptide and dipeptide ester suggests that it can bind divalent (i.e. having two charges like Ca++, Mg++, Zinc++, etc.) metal ions, and in some cases increase solubility or increase blood and body fluid concentrations of the useable form of the metal.

In a preferred embodiment, the current invention may utilize a pharmaceutically effective amount of aspartame as a food/nutrition additive to increase blood/body fluid calcium levels to maintain bone structure or aid in bone density, strength and/or growth development. It is also contemplated that the current invention may utilize a pharmaceutically effective amount of aspartame as a food/nutrition additive to maintain or increase calcium and other metal ion levels in blood and body fluids for general nutritional benefit in man and animals. It is still further contemplated that a preferred embodiment may utilize a pharmaceutically effective amount of aspartame as a food/nutrition additive to maintain or increase calcium and other metal ion levels in blood and body fluids for correcting health related conditions of acute or chronic ‘calcium stress’, i.e. low calcium levels, caused by pathologic conditions or by drug treatments of diabetes, cancer, aging (osteoporosis), and other therapeutics that affect blood calcium levels/availability negatively.

It is still even further contemplated that a preferred embodiment may utilize a pharmaceutically effective amount of aspartame as a food/nutritional additive to maintain or increase calcium and other metal ion levels in blood and body fluids or correcting natural physiologic conditions such as ‘calcium stress’ during pregnancy, birthing and lactation in humans and animals. In another preferred embodiment of the invention, it is contemplated to utilize a pharmaceutically effective amount of aspartame as a preventative of metabolic calcium deficiency in muscles causing cramps and reduced performance during exercise/stress as in general exercise, sports and athletics.

It is, therefore, contemplated that the current invention may utilize aspartame ingested in the dose range of 0.2-5.0 mM/liter of blood, or other pharmaceutically effective amount, to generally increase useable calcium (i.e. calcium bioavailability). The current invention may utilize aspartame ingested in the dose range of 0.2-5.0 mM/liter of blood, or other pharmaceutically effective amount, to generally reduce the effects or treat the condition known as ‘calcium stress’ in humans and animals.

Furthermore, the current invention may utilize a pharmaceutically effective amount of aspartame to generally reduce the effects or treat the condition known as ‘calcium stress’ during birthing, during lactation, for cancer patients, during cancer treatment, for diabetes patients, during treatments for diseases or conditions requiring drugs that deplete or reduce blood calcium levels , and during treatments for all other diseases or conditions requiring drugs that interfere with blood or tissue utilization of calcium.

Still furthermore, the current invention may utilize a pharmaceutically effective amount of aspartame to generally increase calcium bioavailability, divalent mineral ion bioavailability, calcium absorption, facilitated calcium transport, and facilitated divalent mineral ion transport.

Even further, the current invention may utilize a pharmaceutically effective amount of aspartame to generally treat diseases that affect calcium utilization like insulin (diabetes) and parathyroid hormone defects, maintain bone density, increase bone density, stimulates or activates the bone cells called osteoblasts, inhibits or inactivates the bone cells called osteoclasts, treat degenerative bone diseases like osteoarthritis, treat degenerative bone diseases like osteoporosis, and increase muscle mass. It is also therefore contemplated that a preferred embodiment of the invention may be utilized as animal or human feed supplement.

Still even further, the current invention may utilize a pharmaceutically effective amount of aspartame for a dual use application, product or method of use that may reduce both stroke and heart attacks incidences as well as strengthen bones, relieve osteoarthritis pain, as well as other beneficial utilities.

### Topical Applications

It is contemplated a pharmaceutically effective amount of aspartame may be utilized for topical application for pain relief such as but not limited to burns (such as but not limited to friction, thermal, UV/sun, radiation, and chemical), blisters, bruises, shingles, skin diseases/diabetes, stings, insect bites, open sores and general skin injury. Furthermore, the invention may generally be utilized to reduces itching, rash, edema (redness and swelling) and so forth. In a preferred embodiment, a pharmaceutically effective amount of aspartame may be utilized for topical application such as but not limited to lotions in general, body lotions, face creams, foot creams, hand creams, gels, soaps, shampoos, and lip gloss. It is also contemplated for use with shaving creams, aftershave for razor burns and irritation for men and women, lotions or creams associated with thermal, UV/sun, and radiation burns as well as talc or body powder for skin treatments for babies and adults.

It is contemplated that the current invention may utilize the active ingredient Aspartame (Aspartyl-Phenylalanine methyl ester,) as a potent inhibitor of both the COX-1 and COX-2 enzymes, i.e. the cyclo-oxygenases, which are key enzymes in the prostaglandin synthesis pathway. The current invention may provide an active compound of aspartame for relieving topical pain, itch, rash, redness and swelling based on known functions of the COX-1 and COX-2 products. It is understood that prostaglandins are particularly important in the pain response from various forms of burns such as thermal, UV/sunburns, and radiation burns.

The inhibition constants (IC50 or 50% inhibition) for aspartame with COX-1 and COX-2 may be in the sub-micromolar range (0.000001 molar), suggesting that concentrations as low as 1 millimolar (0.001 molar) aspartame (i.e. 294 mg/liter or 1000 times the IC50 levels) may be utilized to full or maximal inhibitory of these enzymes in accordance with the current invention. It is understood that these are the enzymes that are inhibited by aspirin at about the same concentration, potency or efficacy. It is, therefore, contemplated aspartame may be equal to or more effective than aspirin for certain forms of pain relief on a molar, i.e. dose level, basis and utilized as such in a preferred embodiment.

In accordance with a preferred embodiment, the invention may utilize a level of inhibition, which may convert to dose ranges as low as 0.2-0.3 grams ASP/liter, i.e. 1000 milliliters of product, as an effective range. It is understood that the molecular weight of aspartame is 294 grams/mole/or 294 grams of aspartame in one liter volume is one Molar. It is contemplated to use a 1% solution in topical skin application, i.e. 1 gram/100 ml. It is further understood that the dose range may be from 0.2-15 grams/100 ml (or 100 grams) of final product.

It is still further contemplated that the current invention may utilize aspartame for contact dermatitis other than poison ivy/oak/sumac applications. In a preferred construction, the current invention may utilize aspartame with many forms of contact dermatitis and skin irritations (including dryness and eczema). A wide range of skin irritants including ingredients in latex, surgical tape, dyes in clothing, fragrances in soaps, etc., as well as irritation from urine and feces, may all be treated by the current invention utilizing aspartame. It is contemplated the invention may utilize aspartame in an effective pharmaceutical amount for reducing pain and injury from burns as well as diabetic skin sores, general skin trauma, injury conditions, hemorrhoids, athlete's foot, and so forth.

It has also been found that the current invention may be utilized to treat burns in general and more specifically, chemical burns such as acid and base burns. It has been found that using an amount of aspartame in a lotion application has relieved the pain associated with a burn produced by the spilling of acetic acid on an individual's leg. It is also contemplated that the current invention may also help the redness of the skin and/or promote healing for the same injury.

The current invention utilizing aspartame may be used in various salves and fluids for treating a wide variety of skin injury/irritation conditions. It is contemplated aspartame may be utilized with body powders and talc for topical application as such. Other embodiments of the invention may utilize aspartame with other active ingredients to enhance efficacy of skin repair and/or healing, such as corticosteroids (cortisone), antibiotics, other NSAIDS, oils for skin dryness/irritations, therapeutic agents specific for eczema, psoriasis, scar tissue, skin repair/aging, and combinations thereof. It is understood that various delivery method are contemplated and the aforementioned should not be considered to limit the invention as such.

It should also be understood that the term topical application should not be considered to limit the current invention. The current invention further contemplates using a pharmaceutical effective amount of aspartame for oral pain and soreness including sore or irritated throats by means of a mouth wash, gargle, toothpaste, gels, lozenge, cream and so forth.

It is also understood that the invention contemplates numerous applications associated with skin diseases, problems and utilities. By further example, it is known when an allergist does skin tests by challenging with known allergens, many times the local skin reaction is very strong and uncomfortable to the patient. It is contemplated that a pharmaceutically effective amount of aspartame may be used or applied once the challenge is completed to sooth or treat skin. Therefore, it is contemplated that the current invention may be used for skin rash, itch and minor pain from allergen challenges to the skin such as used in allergy skin testing.

It is further understood that the current invention may utilized aspartame for skin conditions when corticosteroids may not be effective, for the itch associated with shingles during recovery phase, on skin rash/dermatitis from radiation exposure and treatment, and to relieve the itch associated with psoriasis. It is further understood that numerous formulations utilizing a pharmaceutically effective amount of aspartame are contemplated for the various utilities described herein.

It is still further contemplated that the current invention may be utilized generally as an insect repellent. In a preferred embodiment, an effective amount of aspartame may be utilized as a topical application to deter biting insects, such as but not limited to mosquitoes.

### Rosacea

It is also contemplated a pharmaceutically effective amount of aspartame (Asp-Phe-methyl ester) may be utilized for a topical application for the skin condition or disease, rosacea, which is typically associated with redness, inflammation, spider veins, pimples, and in severe cases, disfigurement. Rosacea is also often referred to as “adult acne”. The inflammatory condition appears often as a noticeable redness on and about the face and affects mainly adults between the ages of 30-60. Rosacea affects an estimated 14 million people in the United States. It is also known to periodically come and go, and to occur more frequently with those of northern European descent. Spicy foods, heat and alcohol are also believed to worsen its symptoms.

A major problem is that the condition gives noses, cheeks and chins a splotchy redness and an overgrowth of tissue that can be mistaken as stemming from substance abuse or other health issues. This has obvious possible psychological effects to people who not only have their personal appearance lessened, but also the association with being possible alcoholics.

A common treatment currently used is antibiotics. Although rosacea is not caused by bacteria, some antibiotics have been found to reduce the associated symptoms. Many physicians, however, do not prefer to use antibiotics as a form of treatment because of the potential to create more bacterial resistance. Although the condition has been described as quite treatable, the consensus, until very recently, has been that the underlying cause was still unknown.

A recent report from Dr. Richard Gallo, chief of dermatology at UC San Diego, and several colleagues at UCSD, the VA San Diego Healthcare System and institutions in Japan and France, suggests that new evidence has moved a step closer to revealing the root cause of rosacea, or at least what is believed to provoke it to becoming noticeable. Gallo described it as a one-two punch from two proteins—a peptide and an enzyme that may work together to produce a third protein that disfigures the skin. Although the three-protein process may not be the primary cause, it may explain why rosacea turns from mild to worse.

For the study, samples of skin were taken from 20 patients with chronic rosacea in an area near the nose. It was then compared with similar samples from 20 people who did not have rosacea. It was found that skin harbors a peptide called cathelicidin, which normally protects it against infections. People with rosacea may have too much cathelicidin causing an interaction that harms their skin instead. The study found the 20 patients with rosacea to have far more peptides than normal. Those with rosacea also had greatly elevated levels of stratum corneum tryptic enzyme, or SCTE. It is believed the enzyme is what turns abundant amounts of cathelicidin from a protective agent into a harmful one. To further test this theory, Gallo injected mice with cathelicidin peptides found in patients with rosacea and added SCTE. This combination caused the mice to a have a blotchy red appearance.

It is now believed that the skin inflammation in Rosacea is caused by the action of a serine protease called Stratum Corneum Tryptic Enzyme (SCTE). This enzyme breaks down (i.e. cleaves) a protein called cathelicidin. Some of these active breakdown products or protein fragments are claimed to build-up and cause the local inflammation in the skin known as Rosacea. Evidence was provided showing that several specific enzyme inhibitors (a commercial protease inhibitor mix from Roche, Aprotinin, and 4-(2-aminoethyl)-benzenesulfonyl fluoride (AEBSF)) were effective in inhibiting this enzyme. These enzyme inhibitors help to define the enzyme as having trypsin-like specificity. It is believed that these inhibitors effectively blocked skin enzyme activity including SCTE. It is also known that specific antibiotics have been used effectively to treat Rosacea, but only if the antibiotic also had anti-inflammatory activity.

It is contemplated to utilize a pharmaceutically effective amount of FUT-175 which is also known as the trademarked name FUTHAN or Nafamostat Mesilate which has a chemical name of 6-amidino-2-naphthyl p-guanidinobenzoate dimethanesulfonate, hereinafter generally referred to as FUTHAN. The federal trademark registration describes FUTHAN as “pharmaceutical preparations, namely protese [sic] inhibitors for use in the treatment of acute pancreatitis, disseminated intravascular coagulation, vasospasm and septic shock, and for use as an anticoagulant of blood during such as hemodialysis, hemofiltration and cardiopulmonary bypass”. The technical data sheet for FUT-175 (FUTHAN) describes it as a synthetic, broad-specificity protease inhibitor and an inhibitor of the classical and alternate pathways of complement activation.

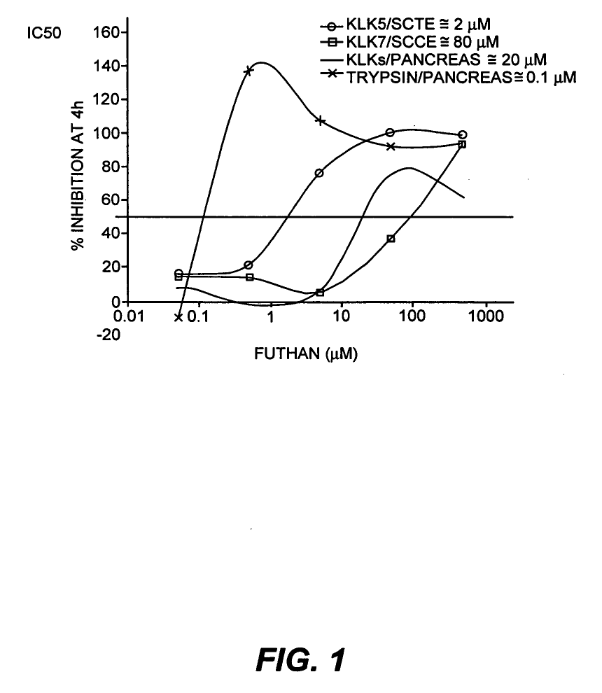

Now referring to FIG. 1, the claim that FUTHAN (FUT-175, Nafamostat, 6-amidino-2-naphthyl-4-guanidinobenzoate) inhibits the skin kallikreins called KLK5(Kallikrein 5)/SCTE (stratum corneum tryptic enzyme) and KLK7 (Kallikrein 7)/SCCE(stratum corneum chymotryptic enzyme) is supported by the data provided. The claim that FUTHAN is an effective treatment for Rosacea derives from this supporting data. It is understood that aspartame, FUTHAN, and combinations thereof may be utilized.

Literature indicates that FUTHAN has a wide specificity for serine proteases (Niinobe et al. FEBS: 172 (#2), July 1984 & Fujii et al. Biochim Biophys Acta: 661 (2) 342-345, 1981.). The list of proteases inhibited by FUTHAN includes pancreatic kallikrein as well as trypsin but not chymotrypsin or enzymes with chymotrypsin specificity. Enzyme inhibition data was collected for KLK5/SCTE, KLK7/SCCE7, pancreatic kallikrein and trypsin (control). The IC50 results (i.e. 50% inhibition) for FUTHAN indicated that the approximate IC50 values for these enzymes were as follows: KLK5/SCTE=2 micromolar, KLK7/SCCE=80 micromolar, pancreatic kallikrein=20 micromolar and trypsin=0.1 micromolar. These results demonstrate a wide variation between the inhibitory function of FUTHAN for different enzymes and this illustrates the inability to predict that FUTHAN will be an effective inhibitor of an enzyme until the assay and data as shown above was done.

Another key observation taken from this data is that FUTHAN was an effective (i.e. micromolar range) inhibitor of the kallikrein KLK7/SCCE having chymotryptic specificity, a fact that could not be known without testing the effect on the actual enzyme. Therefore, the claim that FUTHAN may be an effective treatment of Rosacea based on the inhibition of both key enzymes (KLK5/SCTE and KLK7/SCCE) is confirmed by the data and this claim could not have been validated without evidence of inhibition of these specific enzymes.

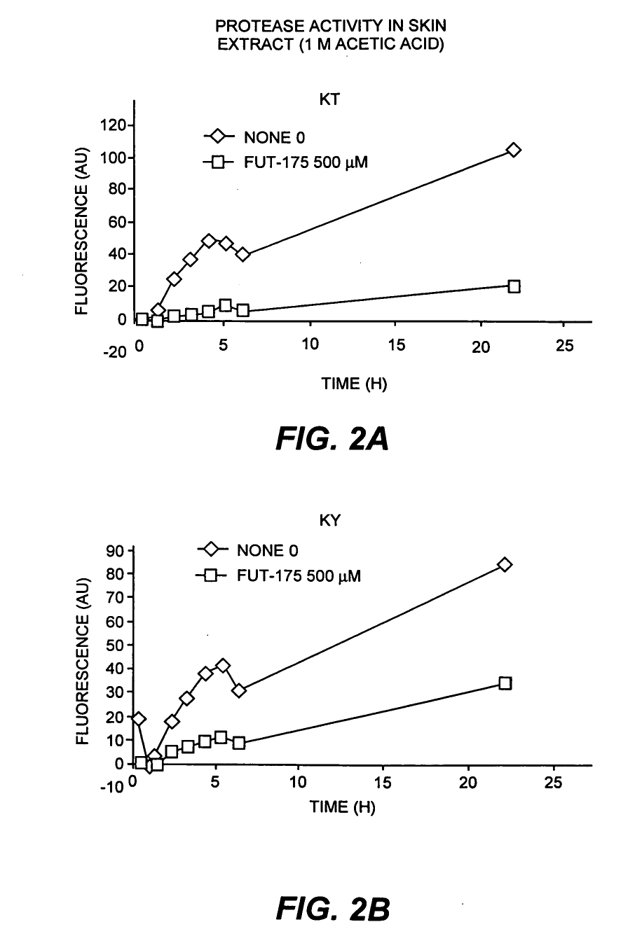

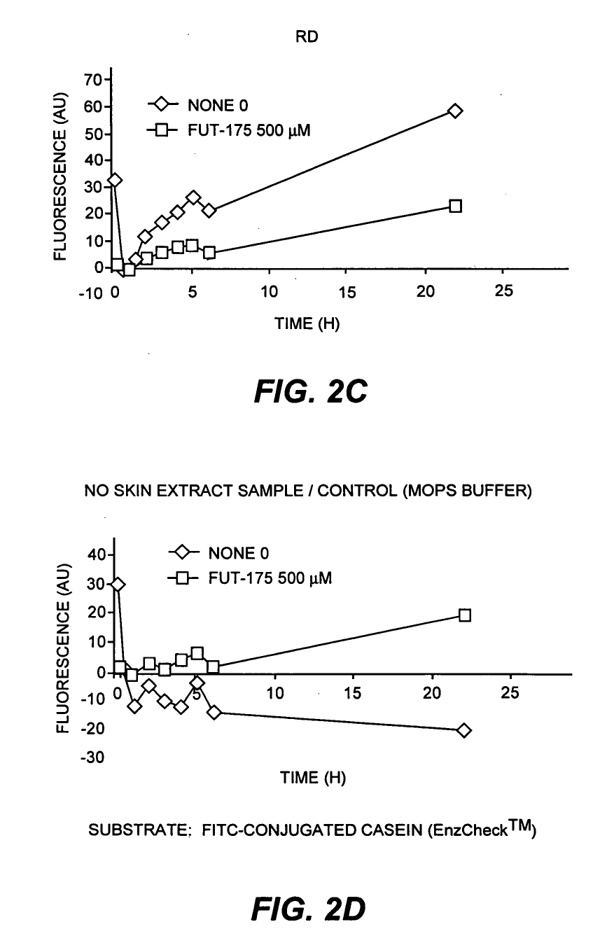

Additional data has been obtained for FUTHAN inhibition of total protease activity in skin extracts from three individuals (KT, KY and RD). This result indicates efficacy of the inhibitor FUTHAN on skin enzymes. These data further support the claim that FUTHAN inhibits the skin enzymes responsible for the condition called Rosacea. See FIG. 2.

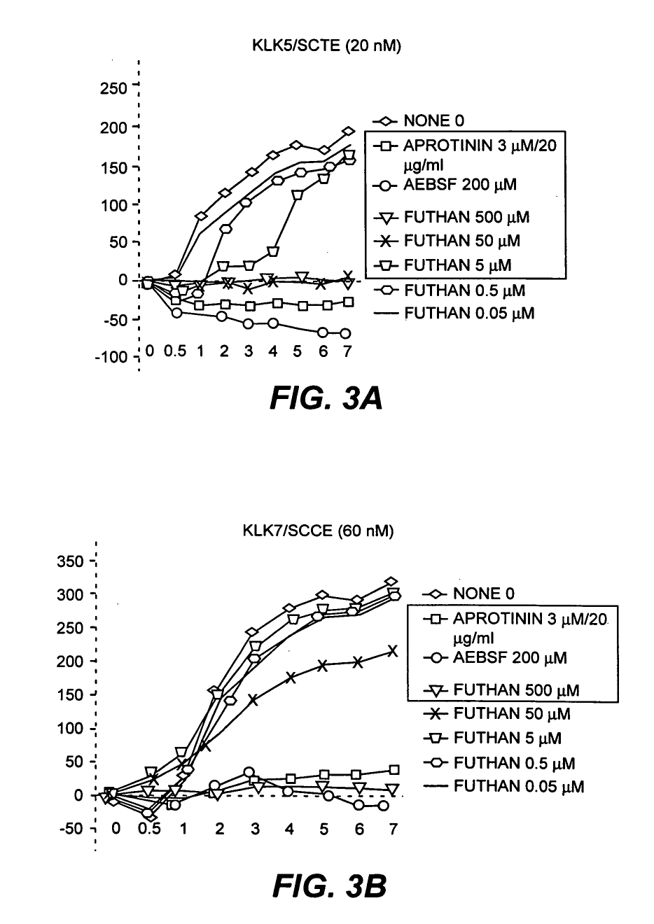

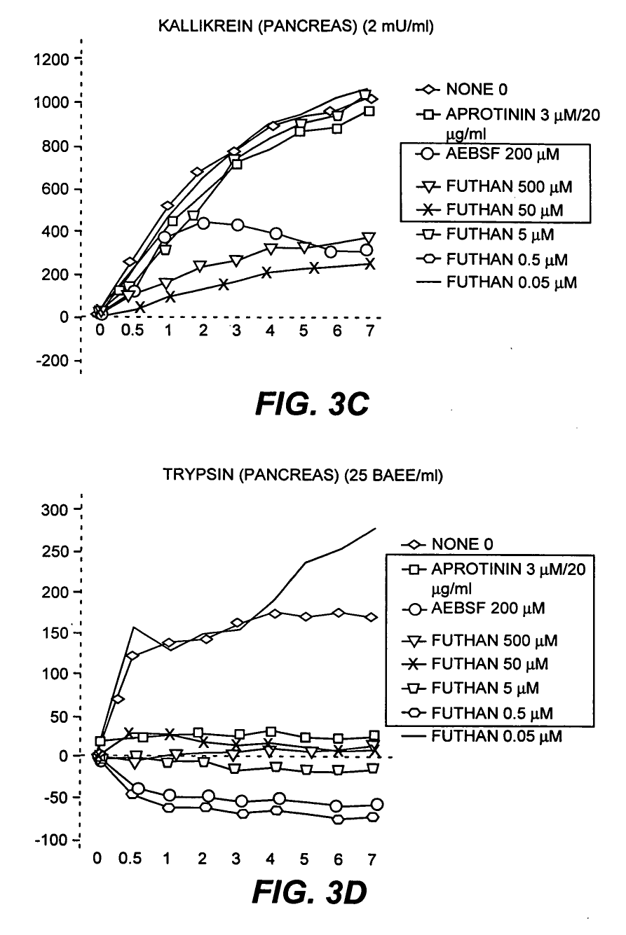

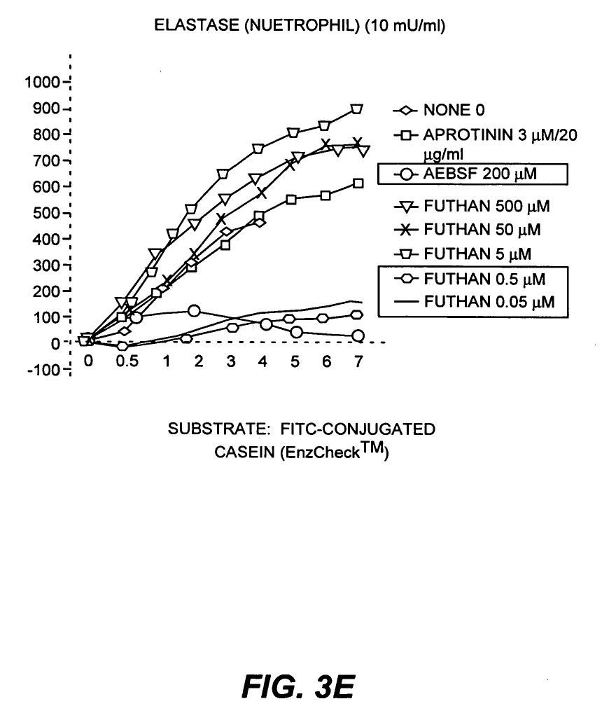

Additional data has been obtained for FUTHAN inhibition of various serine proteases compared with known inhibitors aprotinin and AEBSF. Although FUTHAN is a ‘weaker’ inhibitor than these known serine protease inhibitors, FUTHAN is much less toxic and could be used as a topical treatment while these other compounds have known serious side effects and could not be used effectively. See FIG. 3.

FUTHAN effective dose range on skin may be broad depending on the number of applications and frequency of application. A range between 0.1-500 micromolar FUTHAN in topical lotion, gel or other vehicles may be used. A single application would require a higher dose (i.e. concentration) of FUTHAN than a multiple application protocol where a lower dose would be required. The skin kallikrein data in FIG. 1 supports these dose ranges.

Treatment may be most effective if the inhibitor is used over time including multiple applications a day and applications over several days or weeks. The mechanism of the inhibitor action/function suggests that preventive treatment should be effective to avoid occurrence or re-occurrence. Therefore, a claim is made that treatment prior to the appearance of rosacea in an individual known to have the skin condition should prevent the condition from occurring or re-occurring.

Another claim that is unique to FUTHAN is the fact that it effectively inhibits skin enzymes (kallikrein) with both trypsin and chymotrypsin specificity. This is an unusual property for a protease inhibitor because inhibitors generally inhibit one class of enzymes or enzymes with one substrate specificity. A key trait of FUTHAN is that only one compound and not two compounds are needed to inactivate two kallikreins of different specificity.

It is therefore contemplated the current invention may combine the analgesic anti-inflammatory agent aspartame (Asp-Phe-methyl ester) and a protease inhibitor to effectively treat Rosacea. The current invention provides a unique combination of agents needed to address both the symptoms of the inflammatory skin reaction and the underlying mechanism that causes the skin reaction to occur.

In accordance with a preferred construction, the current invention may provide a formulation comprising a solution, lotion, cream, and/or other delivery means containing about 0.1-1.0% w/vol. aspartame and 0.1-1.0% of a serine protease inhibitor specific to and for tryptic and chymotrytic enzymes. It is understood that this amount should not be considered to limit the current invention and that more or less amounts may be utilized. These inhibitors may include the two inhibitors, Aprotinin and AEBSF. There are many trypsin-specific and chymotrypsin serine protease inhibitors that may be used in combination with Aspartame for this application, including but not limited to peptide analog chloromethylketones (CK) such as Ala-Ala-Lys-CK, Ala-Ala-Arg-CK, Tosyl-Lysine-CK, Tosyl-Arginine-CK and other Lysyl and Arginyl CK analogs.

It is also contemplated that other inhibitors of enzymes having serine protease specificity called SERPINS that are plasma and pancreatic proteins like C1 inhibitor, alpha-1-proteinase inhibitor (alpha-1-anti-trypsin), and anti-thrombin III may be utilized in combination with aspartame to treat rosacea although it is possibly acknowledged that these and other large (i.e. high molecular weight) inhibitors, including the plant inhibitors like soybean trypsin inhibitor, may not penetrate the skin layers as desired. Other low molecular weight and highly potent inhibitors such as phenylmethanesulfonyl fluoride (PMSF) or diisopropyl fluorophosphate (DFP) may also be utilized with the possible acknowledgment that these may be relatively toxic.

The current invention may utilize the class of broad spectrum low molecular weight protease inhibitors designed specifically to inhibit serine protease enzymes in combinations with an aspartame application. An example of such an inhibitor is called FUTHAN (6-amidino-2-naphthyl-p-quanidinobenzoate dimethanesulfonate, FUT-175 or nafamostat mesilate). It is contemplated that this trypsin inhibitor may be utilized because it has been used internally for pancreatitis, and consequently may be known as previously safe for topical applications. It is also contemplated that FUTHAN could be used safely and may effectively penetrate into deeper skin layers to deliver its inhibitory function.

Thus, a preferred embodiment of the current invention may utilize a skin care formulation containing a combination of 0.1-1.0% (wt./vol) of Aspartame and 0.1-1.0 mg FUTHAN/ml (or other protease inhibitor) of a solution, lotion, cream and or other delivery means that may treat the inflammatory reaction and may prevent or reduce the protease-generated skin injury of rosacea.

It is also understood that these amounts should not be considered to limit the current invention as such and that more or less amounts may be utilized. It is still further contemplated the current invention may utilize a pharmaceutically effective amount of aspartame with any other or newly designed serine protease inhibitor that shows inhibitory specificity for SCTE.

### Aspirin Substitute

As generally discussed above, it is further contemplated that the current invention may be utilized as an aspirin substitute wherein in the beneficial attributes of aspirin are realized without some or all of the detrimental attributes. The term aspirin is typically a reference to the drug that reduces pain, fever, inflammation, and blood clotting. Aspirin, a human-made, synthetic version of salicylic acid, generally belongs to the family of drugs called nonsteroidal anti-inflammatory agents. It is also being studied in cancer prevention. Further, aspirin may generally fall into the medication group of “anti-platelet” drugs, which may reduce the chance of stroke, heart attack, and other illnesses in patients with vascular disease. It is contemplated that the invention may also provide the same known and theorized effects as aspirin without some or all of detrimental side effects. It is therefore contemplated the current invention may provide blood thinning abilities which may reduce stroke and heart attack events.

The current invention may utilize a pharmaceutically effective amount of aspartame and or Asp-Phe to generally exhibit analgesic activity. Further, the current invention may utilize a pharmaceutically effective amount of aspartame or Asp-Phe to generally exhibit analgesic activity, which may be delivered in a coated tablet or pill to avoid hydrolysis and inactivation in the stomach and wherein a coated pill or tablet may enhance its analgesic activity. Also, the current invention may utilize a pharmaceutically effective amount of aspartame to generally inhibit cyclo-oxygenase enzymes 1 and 2, i.e. COX-1 and COX-2, and be a functional substitute for aspirin and other NSAIDs that does not cause injury to the lining of the stomach.

The current invention may utilize a pharmaceutically effective amount of aspartame that exhibits a defined set of physiological activities and functions while its immediate metabolite exhibits a separate independent set of physiological activities and functions.

The current invention may utilize a pharmaceutically effective amount of aspartame or Asp-Phe that exhibits dual or multiple physiologic and therapeutic functions.

The current invention may utilize a pharmaceutically effective amount of aspartame or Asp-Phe that the dual or multiple physiologic and therapeutic benefits, functions and activities of aspartame/Asp-Phe can not be separated or divided.

The current invention may utilize a pharmaceutically effective amount of aspartame or Asp-Phe that can both prevent or reduce stroke and heart attacks and enhance bone development, maintenance and growth.

The current invention may utilize a pharmaceutically effective amount of aspartame or Asp-Phe for the treatment of sickle cell anemia and or chronic anemia in general. It has been found that chronic anemia patients have more energy and less muscle pain when using an embodiment of the current invention further including a fruit flavored drink combination with aspartame. It is also contemplated that the current invention may be utilized as a food supplement product with beneficial attributes to chronic anemia patients which may include most cancer patients under treatment.

### Addressing the Potential Side Effects of Aspartame

Besides addressing the very real problem of Phenylketouria, and indicating that Aspartame should not be used by individuals with this condition, there is a major problem associated with claims of Aspartame being dangerous. The main danger that is cited is that the breakdown product methanol can be converted (oxidized) to formaldehyde and large quantities of formaldehyde can be toxic. These are not biochemically sound assertions and clinical studies have widely refuted these claims, however the damage has been done in terms of the general public's perception of the compound.

The body has several metabolic pathways to deal with both methanol and formaldehyde, which is found in many foods and are essential at normal dietary levels. A major pathway is called the “single carbon metabolism pathway” and it is an essential pathway for building both protein (i.e. certain amino acids) and nucleic acids. In fact, when the single carbon pathway dysfunctions humans become anemic.

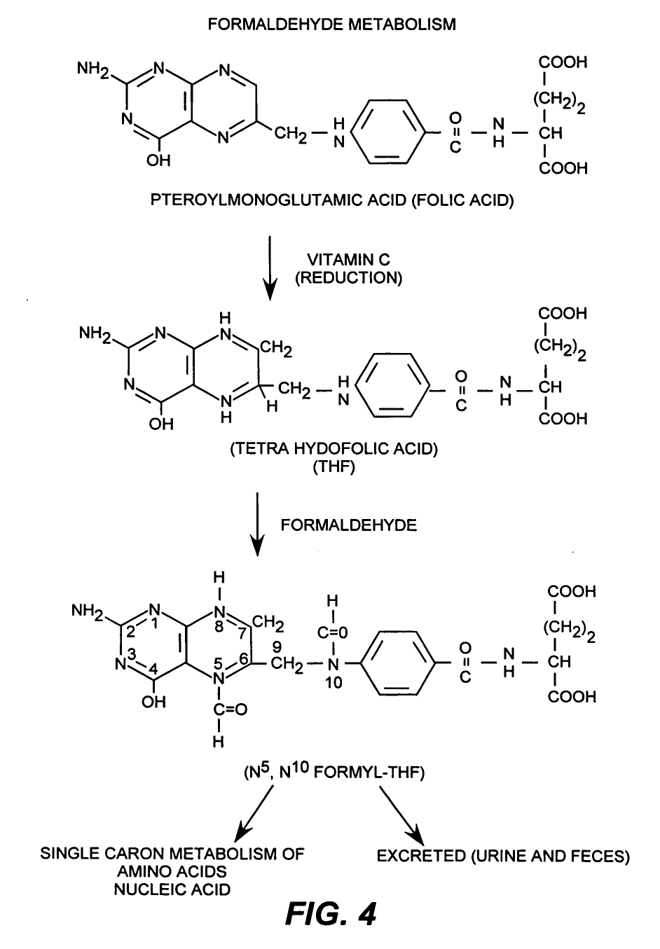

The single carbon pathway actually utilizes formaldehyde that is primarily made in the body. The process involves a vitamin called Folic Acid. The folic acid is reduced to Tertrahydrofolic Acid by reducing agents such as ascorbic acid or Vitamin C. See FIG. 4. In the tetrahydrofolic acid, this compound chemically reacts with formaldehyde at two sites, the N5 and N10 positions. This effectively removes formaldehyde from the body or from embodiment of the invention and the body uses it either for the metabolism of other body molecules or it is excreted. In either case, this is a method to detoxify both methanol and formaldehyde.

It is therefore contemplated the above embodiments may utilize the addition of ascorbic acid (vitamin C) and folic acid along with aspartame. It is contemplated this may keep the levels of methanol and formaldehyde low and avoid any side effects from the normal breakdown of dipeptide ester. Since both of these ingredients are inexpensive, it may be a convenient way to effectively neutralize the urban myths about aspartame being potentially dangerous.

A number of implementations have been described herein. Nevertheless, it will be understood that various modifications may be made. Accordingly, other implementations are within the scope of the following claims. Changes may be made in the combinations, operations, and arrangements of the various parts, elements, and amounts described herein without departing from the spirit and scope of the invention.

We claim:

1. A method for treating rosacea in a patient comprising administering to a patient suffering from rosacea a pharmaceutically effective amount of aspartame.

2. A method for treating osteoporoses in a patient comprising administering to a patient suffering from osteoporoses a pharmaceutically effective amount of aspartame.

3. A method of substituting aspartame for aspirin in a patient comprising administering to a patient a pharmaceutically effective amount of aspartame instead of aspirin.
